(19) United States
(12) Patent Application Publication
(10) Pub. No.: US 2012/0029089 A1
(43) Pub. Date: Feb. 2, 2012

(54) PREPARING BIODEGRADABLE HYDROGEL FOR BIOMEDICAL APPLICATION

(76) Inventors: Chih-Chang Chu, Ithaca, NY (US); Sin-hee Kim, Ithaca, NY (US)

(21) Appl. No.: 13/144,193

(22) PCT Filed: Jan. 14, 2010
(86) PCT No.: PCT/US10/00086

§ 371 Date: Sep. 21, 2011

Related U.S. Application Data
(60) Provisional application No. 61/193,970, filed on Jan. 14, 2009.

(51) Int. Cl. A61K 47/32; A61K 8/73; C08F 2/50

(52) U.S. Cl. 514/772.5; 522/63; 523/105

(57) ABSTRACT

Biodegradable dextran based hydrogel suitable for biomedical application is produced by subjecting polysaccharide substituted with unsaturated moiety, e.g. dextran methacrylate, in aqueous medium to UV or visible light irradiation in the precense of riboflavin/L-arginine or riboflavin/chitosan to cause photocrosslinking of polysaccharide substituted with unsaturated moiety.

### CROSS-REFERENCE TO RELATED APPLICATION

This application claims the benefit of U.S. Provisional Application No. 61/193,970, filed Jan. 14, 2009, the whole of which is incorporated herein by reference.

### BACKGROUND OF THE INVENTION

Preparation of biodegradable hydrogels based on polysaccharides is known. For use in the body, dextran-based hydrogels are preferred since dextran breaks down in the body to glucose which in blood is nutritious and provides energy.

Typically polysaccharide is converted to a hydrogel by subjecting polysaccharide substituted with unsaturated moiety, e.g. dextran methacrylate, in aqueous medium to photoirradiation e.g. UV irradiation, in the presence of photoinitiator to cause polymerization of the polysaccharide and crosslinking via the unsaturated moiety thereby providing a hydrogel. Popular photoinitiators are those which lead to formation of two free radical species (defined as Type I), e.g. benzoin, or those which undergo hydrogen abstraction to generate radicals in the presence of electron donor (defined as Type II), the most popular of which is Michlers ketone which includes an amino substituted part and does not require separate addition of electron donor. The conventional synthetic photoinitiators, e.g. benzoin and Michlers ketone, provide cytotoxic hydrogels and therefore are counterindicated for biomedical application.

Moreover, UV irridation provides risk of damage to skin and eyes.

### SUMMARY OF THE INVENTION

It is an object of this invention to provide biodegradable dextran-based hydrogels for biomedical application which are free of synthetic photoinitiators and synthetic electron donors and which can be produced using UV irradiation or alternatively can be produced using visible light irradiation. The method of this invention is directed to preparing a biodegradable dextran-based hydrogel suitable for biomedical application that is free of synthetic photo-initiators comprising the step of subjecting polysaccharide substituted with unsaturated moiety in the presence of a photoinitiation effective amount of riboflavin and electron donating effective amount of arginine especially L-arginine, or chitosan, to photo-irradiation to cause polymerization and cross-linking of the polysaccharide substituted with unsaturated moiety and formation of hydrogel. The source of the arginine can also be arginine substituted poly (ester amide) as described in “Biodegradable arginine-based poly(ester-amide)s as non-viral

gene delivery reagents” in Biomaterials 29 (2008) 3269-3277, the whole of which is incorporated herein by reference.

As used herein UV irradiation means irradiation of wave length less than and bounding 400 nm down to 10 nm, preferably 350-390 nm, e.g. emitted from a 365 nm long-wave UV lamp (Model XX-15S, 115V, 60 Hz, 0.68 Amp, serial no. 95-0042-05. CE, 15W, UVP, Upland, Calif.).

As used herein visible light irradiation means radiation having a wavelength ranging from 400-700 nm, e.g. emitted by a part or full spectrum lamp, e.g. radiation emitted by a fluorescent lamp (17 watts, Ecolux, FI718-SP-35-ECO, Canada).

### DETAILED DESCRIPTION

The polysaccharide preferably has a weight average molecular weight ranging from 30,000 to 200,000 grams per mole as determined by GPC. The polysaccharide is preferably dextran which preferably has a weight average molecular weight ranging from 64,000 to 76,000 grams per mole as determined by GPC and product information; however, the molecular weight can be any such as to dissolve in solvent or water.

Other useful polysaccharides include, for example, amylose, glycogen, cellulose, chitin, inulin, agarose, zylans, mannan and galactans.

The unsaturated moieties are provided by reaction of polysaccharide in the presence of nucleophilic catalyst, e.g. triethylamine, with methacrylic anhydride, acrylic anhydride, 2-phenylacrylic anhydride, 2-chloroacrylic anhydride, 2-bromoacrylic anhydride, itaconic anhydride, maleic anhydride and styrene maleic anhydride.

A preferred polysaccharide substituted with unsaturated moiety is dextran methacrylate. A degree of substitution of 0.287 was obtained in work described herein using 0.3 molarity methacrylic anhydride. Higher molarity methacrylic anhydride gives higher degree of substitution and lower molarity methacrylic anhydride gives lower degree of substitution. Higher degree of substitution gives more crosslinking on photoirradiation in the presence of photoinitiator and stronger hydrogel, i.e. not broken easily without force, and lower degree of substitution gives less crosslinking on photoirradiation in the presence of photoinitiator and a hydrogel more easily broken. It was found that dextran methacrylate with degree of substitution >0.6 was insoluble in water and therefore unuseful for hydrogel production. It was concluded that degree of substitution of 0.287 gave good balance for water solubility of precursor and resistance to breaking in hydrogel. The degree of substitution for dextran methacrylate ranges, for example, from 0.08 to 0.60.

The photoirradiation to cause polymerization and crosslinking of the dextran methacrylate is suitably carried out in aqueous medium e.g. aqueous phosphate based buffer media (pH-7).

The photoinhibitor used is -(−) riboflavin, also known as vitamin B2, e.g. obtainable from commercial sources, e.g. Sigma-Aldrich of St. Louis, Mo.

As indicated above an election donor for riboflavin is arginine, preferably L-arginine which is the L-form of the naturally occurring arginine which is one of the 20 most common natural amino acids. The source of the arginine can also be arginine substituted poly (ester amide). The election donor can also be chitosan.

When UV irradiation is the photo-irradiation, the photoinitiation effective amount of riboflavin ranges preferably from 0.01 to 2%, very preferably from 0.2-2%, by weight of dextran methacrylate. Less than 0.2% riboflavin by weight of dextran methacrylate gives the disadvantage of a long gelation time. More than 2% riboflavin by weight of dextran methacrylate gives the disadvantage of increasing opacity which can reduce the light penetration for proper gelation in the interior of the sample and increasing gelling time. However, hydrogels have been obtained even with up to 20% riboflavin by weight dextran methacrylate.

When visable light irridation is the photo-irradiation, the photoinitiation effective amount of riboflavin ranges from preferably from 0.01 to 2%, very preferably from 0.01 to 0.51% by weight of the dextran methacrylate. Less than 0.01% riboflavin by weight of dextran methacrylate gives the disadvantage of a long gelation time. More than 0.51% riboflavin by weight of dextran methacrylate gives the disadvantage of increasing opacity which reduces the light penetration into the interior of the solution for proper gelation and increasing gelation time.

When UV irradiation is the photo-irradiation, the election-donating effective amount of L-arginine ranges from 0.8 to 1.6% by weight of the dextran methacrylate for example from 0.8 to 1.2%, by weight dextran methcrylate. Less than 0.8% L-arginine by weight of dextran methacrylate gives the disadvantage of brittleness. More than 1.2% L-arginine by weight of the dextran methacrylate e.g. 1.6% by weight of dextran gives the disadvantage of longer gelation time and the stability of the gel formed was not good because it is too sticky to be handled appropriately. When visible light is the photo-irradiation, arginine was functional in all concentrations by weight of dextran methacrylate and is preferably is present at a concentration ranging from 1 to 20% by weight of dextran methacrylate.

When chitosan is used as the electron donor, it is used, for example in an amount ranging from 0.01 to 2.00% by weight of dextran methacrylate.

Hydrogel formation is carried out by dissolving the dextran methacrylate precursor in aqueous phosphate buffer media (pH7) to provide dextran methacrylate precursor concentration, for example, at 10-50% w/v %, for example at 25 w/v %. After complete dissolution of the dextran methacrylate in the buffer media, riboflavin is added, stirring is carried out to obtain a homogeneous mixture and arginine is then added into the homogeneous mixture. Stirring is then carried out, for example, at room temperature until a homogeneous solution is formed. The solution can be prepared for irradiation by formation of a 1 mm thickness structure and irradiation is carried out using an irradiation source about 15 cm away. UV irradiation is carried out, for example, using a 365 nm long-wave UV lamp (Medel XX-15S, 115V, 60 HZ, 0.68 Amp, serial No. 95-0042-05, CE, 15W, UVP, Upland, Calif., Visible light irradiation is carried out, for example, using a florescence lamp (17 watts, Ecolox, F 1718-SP-35-ECO, Canada). Gelation is complete when the whole gel can be lifted without any fluid flowing.

Swelling property of hydrogels is important because it indicates amount of solution absorbed by the hydrogel relative to dried hydrogel.

The degree of swelling can be characterized by swelling ratios (%). These can be calculated as described in Kim, S. H., et al, Journal of Biomedical Materials Research Part B, Applied Biomaterials 2009, page 390-400 (published online 10 Jun. 2009) and Kim, S.-H., Fibers and Polymers 2009, Vol 10, No. 1, 14-20.

Testing showed that hydrogels produced herein using UV irridation provided 80% swelling ratio independent of pH of swelling test medium and that hydrogels produced herein using visible light irridation gave about 70% swelling ratio with pH 7 test medium and up to 100% swelling ratio with pH3 and pH10 test medium and 0 to 200% independent of pH.

For example, a method for preparing a toxicity free biodegradable hydrogel from dextran methacrylate where the dextran has a weight average molecular weight ranging from 30,000 to 200,000 grams per mole, for example from 64,000 to 76,000 grams per mole, and has a degree of substitution ranging from 0.08 to 0.60, comprises subjecting the dextran methacrylate in the presence of photo initiation effective amount of riboflavin ranging from 0.2-2% by weight of dextran methacrylate and an electron donating effective amount of L-arginine ranging from 0.8 to 1.6% for example 0.8 to 1.2% by weight of dextran methacrylate, at pH 1.0 to 10.0 to gelling effective amount of UV irradiation, to produce biodegradable dextran based toxicity free hydrogel with a swelling ratio ranging from 0 to 200% independent of pH.

In another case, a method herein is for preparing a toxicity free biodegradable hydrogel from dextran methacrylate where the dextran methacrylate has a weight average molecular weight ranging from 30,000 to 200,000 grams per mole, for example, from 64,000 to 76,000 grams per mole and has a degree of substitution ranging from 0.08 to 0.60, and comprises subjectiving the dextran methacrylate in the presence of a photoinitiating effective amount of riboflavin ranging from 0.01 to 0.51 percent by weight of the dextran methacrylate and an electron donating effective amount of L-arginine ranging from 1 to 20% by weight of dextran methacrylate at pH of 1 to 10 to visible light irradiation to produce a biodegradable dextran based toxicity free hydrogel with a swelling ratio ranging from 64 to 98% with the swelling ratio being higher at alkaline or acid pH than at neutral pH.

In another embodiment the invention is directed to a system for forming hydrogel by UV application or by visible light application.

For UV initiated crosslinking the hydrogel forming system comprises
a) dextran methacrylate having a weight average molecular weight ranging from 30,000 to 200,000 grams per mole and a degree of substitution ranging from 0.08 to 0.60;
b) a photoinitiation effective amount of riboflavin ranging from 0.2 to 2% by weight of dextran methacrylate, and
c) an electron donating effective amount of L-arginine ranging from 0.8 to 1.2 by weight of dextran methacrylate.

For visible light initiated crosslinking the hydrogel forming system comprises;
a) dextran methacrylate having a weight average molecular weight ranging from 30,000 to 200,000 and a degree of substitution ranging from 0.08 to 0.60;
b) a photoinitiation effective amount of riboflavin ranging from 0.01 to 0.51% by weight of dextran methacrylate, and
c) an electron donating effective amount of L-arginine ranging from 1 to 20% by weight of dextran methacrylate.

In another case, the invention is directed at a biodegradable nontoxic hydrogel including dextran as a polysaccharide moiety, which is free of residual synthetic photoinitiators and contains as residual photoinitiator only that which is activated by visible light.

We turn now to applications for hydrogels described and prepared herein. Bioactive agents e.g. any water-soluble biologically active agent or biologic, e.g. antibiotics, antiflammatory agents, wound healing agents, proteins, growth factors such as fibroblast growth factor, cytokines such as IL-2; 6 and 12, or DNA receptors, can be associated into the hydrogels herein by dissolving or suspending bioactive agent into the solution subjected to photoirradiation herein to provide hydrogels containing bioactive agent thereby functioning as controlled release drug composition. The hydrogels produced herein can also be used as nontoxic water soluble coating on skin and other body parts.

Elements of the invention herein are disclosed, Kim, S.-H., et al, Journal of Biomedical Materials Research Part B: Applied Biomaterials, 2009, pages 390-400 (published online 10 Jun. 2009) and Kim, S.-H., Fibers and Polymers 2009, vol. 10, No. 1, 14-20, the whole of which are incorporated herein by reference.

Example of making precursor material and Working Examples of the invention herein are set forth below.

### Working Example I

### Preparation of Dextran-Methacrylate as a Hydrogel Precursor

Dextran was dissolved in the LiCl/DMF (10 wt. %) solvent system at 90° C. under nitrogen gas purge. After a complete dissolution, the solution was cooled down to 70° C. and triethylamine, as a nucleophilic catalyst, was added slowly. The amount of triethylamine added was 10 mol. % of methacrylic anhydride. The dextran solution was stirred vigorously for 10 min and methacrylic anhydride was then slowly injected into the system with a syringe. The amount of methacrylic anhydride added was 0.3 molarity of the hydroxyl groups in dextran glucose unit. The reaction was conducted for 5 h at 70° C. The dextran methacrylate product in the reaction mixture was precipitated in cold isopropyl alcohol, washed several times with isopropyl alcohol, and dried at room temperature in a vacuum oven.

To remove any residual unreacted methacrylic anhydride in dextran-methacrylate, the dextran-methacylate was dissolved in DMF and precipitated in isopropyl alcohol. The same procedure was repeated for 3 times to achieve a completely purified dextran-methacylate precursor.

A degree of substitution of methacrylic groups to dextran was determined to be 0.287 degree of substitution (D.S.) by the integration and normalization of double bonds in methacrylic segment (5.5˜6.5 ppm) and the hydroxyl hydrogen peaks of dextran backbone (4.3˜5.5 ppm). The maximum D.S. was assigned as 3.00 when all three hydroxyl group was substituted. The following equation was used to calculate D.S. and the equation derivation is discussed in detail in Kim, S.-H., “Synthesis of dextran-based hydrogels, their characterization structural study, and doing control release property, Cornell University, UMI Dissertation Services; 1999: 211-216.

D.S. of methacrylic groups onto dextran=4R/(R+2)

R=B/A
A=Integrated area of hydroxyl hydrogen peaks of dextran backbone (4.3˜5.5 ppm)
B=Integrated area of methacrylic segment (5.5˜6.5 ppm)

### Working Example II

### Photocrosslinking of Dextran-Methacrylate to Make a Hydrogel Using (−)-Riboflavin/L-Arginine as Photoinitiator/Co-Initiator Under UV Irradiation

The dextran-methacrylate of Example I as polymer precursor was dissolved in buffer media of pH 3, 7, and 10, respectively. The polymer precursor concentration was maintained at 25 w/v % in all gel fabrications. After complete dissolution of dextran-methacrylate precursor in a buffer medium, riboflavin was added with the concentrations of 0.2, 1, 2, 4, 12, 20 wt. % of the polymer precursor, respectively. The mixture was stirred for 5 min. until a homogeneous mixture was formed and L-arginine of concentrations of 0.4, 0.8, 1.2, 1.8, and 2.0 weight ratio of dextran-methacrylate precursor was then added. The mixture was subsequently stirred for 5 min. at room temperature until a homogeneous solution was formed. The solution was poured onto a plastic plate to obtain one mm thickness and was irradiated until a complete hydrogel was formed, i.e. using a 365 nm long-wave UV lamp (Model XX-15S, 115V, 60 Hz, 0.68 Amp, serial no. 95-0042-05. CE, 15W, UVP, Upland, Calif., USA) positioned about 15 cm above the one mm thickness structure until a complete hydrogel was formed. The solution was scratched by spatula at intervals and the gel starting time was recorded when the scratch mark of a spatula was permanent. The gelation time was the time when the whole gel could be lifted without any fluid flowing around.

The effect of the riboflavin concentration and pH aqueous reaction medium on dextran-methacrylate hydrogel characteristics as determined in work supporting this patent application is shown in Table 1 below:

TABLE 1

| | | L-arginine (wt. | | | |
|---|---|---|---|---|---|
| | Riboflavin (wt. % of | percent of dextran- | | | |
| pH | dextran-methacrylate) | methacrylate) | Turbidity | Gelₛₜ* | Gel_{fin}** |
| pH 3 | 0.2 | 0.8 | transparent | 30 min. | 60 min. |
| | 1 | 0.8 | transparent | 30 min. | 60 min. |
| | 2 | 0.8 | semi-transparent | 30 min. | 60 min. |
| | 4 | 0.4 | opaque | 30 min. | 60 min. |
| | | 0.8 | opaque | 30 min. | 60 min. |
| | | 1.2 | opaque | 30 min. | 60 min. |
| | | 1.6 | opaque | 30 min. | 60 min. |
| | | 2.0 | opaque | 30 min. | 60 min. |
| | 12 | 0.4 | opaque | 60 min. | 120 min. |
| | | 0.8 | opaque | 60 min. | 120 min |
| | | 1.2 | opaque | 60 min. | 120 min |
| | | 1.6 | opaque | 60 min. | 120 min |
| | | 2.0 | opaque | 60 min. | 120 min |
| | 20 | 0.4 | opaque | — | No gel |
| | | 0.8 | opaque | — | No gel |
| | | 1.2 | opaque | — | No gel |
| | | 1.6 | opaque | — | No gel |
| | | 2.0 | opaque | — | No gel |
| pH 7 | 0.2 | 0.4 | transparent | 5 min. | 30 min. |
| | | 0.8 | transparent | 5 min. | 30 min. |
| | | 1.2 | transparent | 5 min. | 30 min. |
| | | 1.6 | transparent | 5 min. | 30 min. |
| | | 2.0 | transparent | 5 min. | 30 min. |
| | 1 | 0.4 | transparent | 5 min. | 30 min. |
| | | 0.8 | transparent | 5 min. | 30 min. |
| | | 1.2 | transparent | 5 min. | 30 min. |
| | | 1.6 | transparent | 5 min. | 30 min. |
| | | 2.0 | transparent | 5 min. | 30 min. |
| | 2 | 0.4 | semi-transparent | 5 min. | 30 min. |
| | | 0.8 | semi-transparent | 5 min. | 30 min. |
| | | 1.2 | semi-transparent | 5 min. | 30 min. |
| | | 1.6 | semi-transparent | 5 min. | 30 min. |
| | | 2.0 | semi-transparent | 5 min. | 30 min. |
| | 4 | 0.4 | opaque | 30 min. | 60 min. |
| | | 0.8 | opaque | 30 min. | 60 min. |
| | | 1.2 | opaque | 30 min. | 60 min. |
| | | 1.6 | opaque | 30 min. | 60 min. |
| | | 2.0 | opaque | 30 min. | 60 min. |
| | 12 | 0.4 | opaque | 60 min. | 120 min. |
| | | 0.8 | opaque | 60 min. | 120 min. |
| | | 1.2 | opaque | 60 min. | 120 min. |
| | | 1.6 | opaque | 60 min. | 120 min. |
| | | 2.0 | opaque | 60 min. | 120 min. |
| | 20 | 0.4 | opaque | 120 min. | 300 min. |
| | | 0.8 | opaque | 120 min. | 300 min. |
| | | 1.2 | opaque | 120 min. | 300 min. |
| | | 1.6 | opaque | 120 min. | 300 min. |
| | | 2.0 | opaque | 120 min. | 300 min. |
| pH 10 | 0.2 | 0.8 | transparent | 30 min. | 60 min. |
| | 1 | 0.8 | transparent | 30 min. | 60 min. |
| | 2 | 0.8 | semi-transparent | 30 min. | 60 min. |
| | 4 | 0.4 | opaque | 30 min. | 60 min. |
| | | 0.8 | opaque | 30 min. | 60 min. |
| | | 1.2 | opaque | 30 min. | 60 min. |
| | | 1.6 | opaque | 30 min. | 60 min. |
| | | 2.0 | opaque | 30 min. | 60 min. |
| | 12 | 0.4 | opaque | — | No gel |
| | | 0.8 | opaque | — | No gel |
| | | 1.2 | opaque | — | No gel |
| | | 1.6 | opaque | — | No gel |
| | | 2.0 | opaque | — | No gel |
| | 20 | 0.4 | opaque | — | No gel |
| | | 0.8 | opaque | — | No gel |
| | | 1.2 | opaque | — | No gel |
| | | 1.6 | opaque | — | No gel |
| | | 2.0 | opaque | — | No gel |

*Gelation starting time,
**Gelation finishing time.

The effect of L-arginine concentration on physical shape and characteristics of dextran methacrylate hydrogels in pH 7 aqueous reaction medium as determined in work supporting this patent application is shown in Table 2 below:

| L-Arginine (wt. % of | Riboflavin (wt. % of | | | Physical shape |
|---|---|---|---|---|
| dextran-methacrylate) | dextran-methacrylate) | Brittle/Compliable* | Stickiness** | stability*** |
| 0.4 | 0.2 | Brittle | Little sticky | Good |
| | 1 | Brittle | Little sticky | Good |
| | 2 | Brittle | Little sticky | Good |
| | 4 | Brittle | Little sticky | Good |
| | 12 | Brittle | Little sticky | Good |
| | 20 | Brittle | Little sticky | Good |
| 0.8 | 0.2 | Compliable | Sticky | Excellent |
| | 1 | Compliable | Sticky | Excellent |
| | 2 | Compliable | Sticky | Excellent |
| | 4 | Compliable | Sticky | Excellent |
| | 12 | Compliable | Sticky | Excellent |
| | 20 | Compliable | Sticky | Excellent |
| 1.2 | 0.2 | Compliable | Sticky | Excellent |
| | 1 | Compliable | Sticky | Excellent |
| | 2 | Compliable | Sticky | Excellent |
| | 4 | Compliable | Sticky | Excellent |
| | 12 | Compliable | Sticky | Excellent |
| | 20 | Compliable | Sticky | Excellent |
| 1.6 | 0.2 | Compliable | Very sticky | Not bad |
| | 1 | Compliable | Very sticky | Not bad |
| | 2 | Compliable | Very sticky | Not bad |
| | 4 | Compliable | Very sticky | Not bad |
| | 12 | Compliable | Very sticky | Not bad |
| | 20 | Compliable | Very sticky | Not bad |
| 2.0 | 0.2 | Compliable | Very sticky | Bad |
| | 1 | Compliable | Very sticky | Bad |
| | 2 | Compliable | Very sticky | Bad |
| | 4 | Compliable | Very sticky | Bad |
| | 12 | Compliable | Very sticky | Bad |
| | 20 | Compliable | Very sticky | Bad |

*The gel was considered as “compliable” when the formed gel was bent 90° in one way and also bent at the same angle in the opposite way without breaking. A broken gel was determined as “brittle”.
**The gel was considered as “sticky” when it tends to stick to the surface without breaking its physical shape during the handling. The gel was considered as “very sticky” when it tends to stick to the surface so hard causing handling problem.
***In defining the physical shape stability, the following standards were used. “Excellent” means the gel formed was not break under forces. No changes were made during drying and moving processes. “Good” means the gel formed was not break easily without force. No changes were shown during drying and moving processes with caution. “Not bad” means the gel formed was in shape when formed but changed its shape during drying or moving. “Bad” means the gel formed was not in shape when formed because it is very sticky. Therefore, the formed gel changed its shape during drying and moving processes.

For swelling testing, the dextran-methacrylate hydrogels were cut into several pieces and dried in a vacuum oven at a room temperature until no weight change of hydrogel was detected. The dried dextran hydrogel pieces (0.1 g each) were soaked in PBS buffer media of pH 3, 7, and 10 respectively. The soaked hydrogels were removed at the predetermined intervals and weighed until no further weight change was observed. Swelling ratio of the hydrogels was calculated by the following equation. An average of three samples for each condition was recorded.

$$Swelling ratio ( \% ) = \frac{{W}_{s} - {W}_{o}}{{W}_{o}} \times 100$$

$${W}_{s} \text{:} Weight of a swollen hydrogel$$

$${W}_{o} \text{:} Weight of a dried hydrogel$$

The dextran-methacrylate hydrogels showed a moderate swelling property (around 80%). The swelling ratios determined were independent of pH of the swelling test medium. The hydrogels absorbed a majority of the water from a test medium during the first 20 minutes in the test medium and reached an equilibrium thereafter. The swelling ratio did not change after 24 hours of hydrogel in the test medium. Dried hydrogel (approximately 1 cm×1 cm) swelled to 1.3 cm×1.3 cm. The swollen hydrogels were approximately 130% larger than the dried hydrogels.

### Working Example III

### Photocrosslinking of Dextran-Methacrylate to Make a Hydrogel Using (−)-Riboflavin/L-Arginine as Photoinitiator/Co-Initiator Under Visible Light Irradiation

The dextran-methacrylate of Example I as polymer precursor was dissolved in a buffer media (pH 7). The polymer precursor concentration was maintained as 25 w/v %. After the complete dissolution of dextran-methacrylate precursor in a buffer medium, (−)-riboflavin was added over a wide range concentrations from 0.01, 0.1, 0.2, 0.5, 1, 2, 5, 10, to 20 wt. % of dextran-methacrylate precursor. The mixture was stirred for 5 min until a homogeneous mixture was formed. L-arginine of concentrations 0, 1, 2, 5, 10, 20, 40, 60, and 100 wt. % of dextran-methacrylate precursor was then added into the above homogeneous mixture. The mixture was subsequently stirred for 5 min at room temperature until a homogeneous solution was formed. The solution was poured onto a circular Teflon mold to obtain 1 mm thickness and irradiated by a fluorescence lamp (17 watts, Ecolux, FI718-SP-35-ECO, Canada) until a complete hydrogel was formed (15˜40 min.). The distance between the hydrogel precursor solution and the lamp was about 15 cm.

The gelation time was monitored to elucidate the optimum condition for the hydrogel formation upon the irradiation of the visible light. The gel starting point was considered when a scratch mark remained on the hydrogel precursor surface upon scratching with a spatula. The gelation was complete when the whole gel could be lifted without any fluid flowing.

The effect of riboflavin concentration on hydrogel characteristics at 10 wt % L-arginine under visible light irradiation as determined in work supporting this application is indicated below.

The effect of riboflavin concentration on the photocrosslinking of dextran-methacrylate precursor at 10 wt % L-arginine to produce hydrogel at 25 weight dextran-methacry late in pH 7 buffer solution is shown in Table 3 below. The various concentrations of riboflavin, from 0 to 20 wt % of hydrogel precursor, were studied to elucidate the optimum reaction condition for the proposed photoinitiation system using (−)-riboflavin/L-arginine system under a visible light source. Without a (−)-riboflavin photoinitiator, no gelation was observed. One distinctive characteristic in the photocrosslinking using (−)-riboflavin as a photoinitiator is that only minute amounts of riboflavin are needed since large amounts of riboflavin concentration actually did not lead to a hydrogel formation. (−)-Riboflavin of only less than 1 wt. % was sufficient to initiate the photocrosslinking reaction of dextran-methacrylate precursor. A reason that a higher (−)-riboflavin concentration retarded the gelation can be attributed to the increased opacity of the hydrogel precursor solution which could hinder the penetration of the visible light to the precursor solution. This same tendency was also observed in the Working Example II UV light study using riboflavin for the photoinitiation of dextran methacrylate hydrogel precursor. As a result, riboflavin of 1-20 wt. % did not result in a satisfactory hydrogel formation. On the other hand, riboflavin of a very low concentration (0.01 wt. %) led to the formation of dextran methacrylate hydrogels having a good form and shape. The optimum riboflavin concentration in the formation of the dextran methacrylate hydrogel was 0.01-0.5 wt. % in terms of the gelation speed. Within this riboflavin concentration range, the gelation started at 5 min. and completed at 15 min. by the visible light irradiation. The physical shapes were excellent for both 0.1 and 0.2 wt. % riboflavin. Therefore, 0.1-0.2 wt. % riboflavin photoinitiator concentration is the best reaction condition in the photocrosslinking dextran-methacrylate precursor into hydrogels upon visible light irradiation.

TABLE 3

| Riboflavin | | | | | | Physical |
|---|---|---|---|---|---|---|
| (wt. %) | Gelₛₜ* | Gel_{fin}** | Turbidity | Compliability | Stickiness | shape |
| 0 | — | No gel | — | — | — | — |
| 0.01 | 5 min. | 15 min. | Transparent | Good | Medium | Good |
| 0.1 | 5 min. | 15 min. | Transparent | Good | Medium | Excellent |
| 0.2 | 5 min. | 15 min. | Transparent | Good | Medium | Excellent |
| 0.5 | 5 min. | 15 min. | Transparent | Good | Medium | Good |
| 1 | 30 min. | Incomplete | Transparent | — | — | — |
| 2 | 30 min. | Incomplete | Semi-transparent | — | — | — |
| 5 | — | No gel | opaque | — | — | — |
| 10 | — | No gel | opaque | — | — | — |
| 20 | — | No gel | opaque | — | — | — |

*Gelation starting time.
**Gelation completion time.

As used in Table 3, turbidity means degree of opacity.

As used in Table 3, compliability mean the same as for Table 2.

As used in Table 3, medium stickness means stick to the skin or other surface and can be removed without any damage of the hydrogel like an post-it note.

As used in Table 3, good physical shape means the same for Table 2.

As uses in Table 3, excellent physical shape means the same for Table 2.

The effect of L-arginine concentration on the photocrosslinking on the of dextran-methacrylate precursor at 0.1 weight % riboflavin in 25 weight % dextran-methacrylate precursor in pH 7 buffer solution is shown in Table 4 below.

L-arginine promoted the photocrosslinking reaction of (−)-riboflavin in all concentrations. However, too high concentration of L-arginine, between 40-100 wt. % of dextran-methacylate precursor lengthened the gelation completion time. The solution with this concentration range of L-arginine was too sticky and viscous for hydrogel to be formed in 25 minutes or less (for example in 15 minutes). The hydrogel was very quickly formed with 1-20 wt. % L-arginine. The gelation started at 5 min and completed after 15 min. The optimum concentration of L-arginine for achieving dextran methacrylate hydrogels having adequate structural integrity and strength during physical handling was considered to be from 5 to 10 wt %. This physical property is very important in the biomedical application, such as a wound healing system.

TABLE 4

| L-arginine | | | | | | Physical |
|---|---|---|---|---|---|---|
| (wt. %) | Gelₛₜ* | Gel_{fin}** | Turbidity | Compliability | Stickiness | shape |
| 0 | 5 min. | 15 min. | Transparent | Not good | Not sticky | Good |
| 1 | 5 min. | 15 min. | Transparent | Not good | Not sticky | Good |
| 2 | 5 min. | 15 min. | Transparent | Not bad | Little | Excellent |
| | | | | | sticky | |
| 5 | 5 min. | 15 min. | Transparent | Good | Sticky | Excellent |
| 10 | 5 min. | 15 min. | Transparent | Good | Sticky | Excellent |
| 20 | 5 min. | 15 min. | Transparent | Good | Sticky | Good |
| 40 | 5 min. | 30 min. | Transparent | Very good | Very | Not bad |
| | | | | | sticky | |
| 60 | 5 min. | 40 min. | Transparent | Too compliable | Too sticky | Not good |
| 100 | 5 min. | 40 min. | Transparent | Too compliable | Too sticky | Not good |

*Gelation starting time.
**Gelation completion time.

As used in Table 4, turbidity means degree opacity.

As used in Table 4, compliability, degrees of sticking and characterization of physical shape are the same as for Table 2.

For swelling testing, the dextran-methacrylate hydrogels were cut in to several pieces and dried in vacuum over at room temperature until no weight change of a hydrogel was detected. The dried hydrogel pieces (0.1 g) were soaked in PBS buffer media of pH 3, 7 and 10, respectively at room temperature. The soaked hydrogels were removed at predetermined intervals and weighed until no further weight change was observed. Swelling ratio of the hydrogels was calculated by the following equation. An average of three samples of each condition was recorded.

Swelling ratio(%)={(Wₛ−Wₒ)/Wₒ}×100

where Wₛ: Weight of a swollen hydrogel and Wₒ: Weight of a dried hydrogel

In determining swelling property of dextran-methacylate hydrogel formed by the visible light irradiation in the presence of (−)-riboflavin/L-arginine as photoinitiator/co-initiator, it was determined that most of the water was absorbed in the first 30 min in all pH media and about half of the water was absorbed in the first 10 min (68% in pH 3, 45% in pH 7, and 64% in pH 10). After 30 min, water absorption of the dextran-methacrylate hydrogels reached an equilibrium regardless of the pH of the media. The swelling ratio of dextran-methacrylate hydrogel in pH 7 media showed the lowest value (70%). The swelling ratio of dextran-methacrylate hydrogel in pH 3 media was the highest. The swelling ratio in pH 10 media was found to be a little smaller than in pH 3 media. The hydrogels in acidic and alkaline media started losing their physical integrity and disintegrated after 1 h. However, the hydrogels in the neutral pH media were intact in its physical form after 24.

### Working Example IV

### Photocrosslinking of Dextran-Methacrylate to Make a Hydrogel Using (−)-Riboflavin-Chitosan as Photoinitiator Under Visible Light Irradiation

The dextran-methacrylate of Example I as polymer precursor is dissolved in a buffer media (pH 7). The polymer precursor concentration is maintained as 25 w/v %. After the complete dissolution of dextran-methacrylate precursor in a buffer medium, (−)-riboflavin is added over a wide range concentration of 1 wt. % of dextran-methacrylate precursor. The mixture is stirred for 5 min until a homogeneous mixture is wt % formed. Chitosan of concentration of 1% dextran-methacrylate precursor is then added into the above homogeneous mixture. The mixture is subsequently stirred for 5 min at room temperature until a homogeneous solution was formed. The solution is poured onto a circular Teflon mold to obtain 1 mm thickness and irradiated by a fluorescence lamp (17 watts, Ecolux, FI718-SP-35-ECO, Canada) until a complete hydrogel was formed (15˜40 min.). The distance between the hydrogel precursor solution and the lamp is about 15 cm. Gelation is complete when the whole gel can be lifted without any fluid flowing. A biodegradable nontoxic hydrogel free of synthetic photoinitiators is prepared.

### Variations

The foregoing description of the invention has been presented describing certain operable and preferred embodiments. It is not intended that the invention should be so limited since variations and modifications thereof will be obvious to those skilled in the art, all of which are within the spirit and scope of the invention.

1. A method for preparing a biodegradable hydrogel that is free of synthetic photo-inhibitors comprising the step of subjecting polysaccharide substituted with unsaturated moiety in the presence of a photoinitiation effective amount of riboflavin and electron donating effective amount of arginine or of chitosan to photo-irradiation in aqueous medium to cause polymerization and cross-linking of the polysaccharide substituted with unsaturated moiety.

2. The method according to claim 1 where the photo-irradiation is ultraviolet irradiation.

3. The method according to claim 1 where the photo-irradiation is visible light irradiation.

4. The method according to claim 2 on claim 3 where the polysaccharide is dextran having a weight average molecular weight ranging from 30,000 to 200,000 grams per mole.

5. The method of claim 4 where the polysaccharide substituted with unsaturated moiety is dextran methacrylate.

6. The method of claim 5 where the dextran methacrylate has a degree of substitution ranging from 0.08 to 0.60.

7. The method according to claim 2 where the polysaccharide-substituted with unsaturated moiety is dextran methacrylate having a degree of substitution ranging from 0.08 to 0.60, the photoinitiation effective amount of riboflavin ranges from 0.2-2% by weight of dextran methacrylate, the electron donor is L-arginine electron donating effective amount of L-arginine ranges from 0.8˜1.2% by weight of dextran methacrylate, the method is carried out at pH ranging from 1.0 to 10 and the method of is carried out to produce a toxicity free biodegradable dextran based toxicity free hydrogel with a swelling ratio ranging from 0 to 200% independent of pH.

8. The method according to claim 3 where the polysaccharide substituted with unsaturated moiety is dextran methacrylate having a degree of substitution ranging from 0.08 to 0.60, the photoinitiation effective amount of riboflavin ranges from 0.01 to 0.51 percent by weight of the dextran methacrylate, the electron donor is L-arginine electron donating effective amount of L-arginine ranges from 1 to 20% by weight of the dextran methacrylate, the method is carried out at pH ranging from 1 to 10 and the method is carried out to produce a biodegradable dextran-based, toxicity free hydrogel with a swelling ratio ranging from 64 to 98% with the swelling ratio being higher at alkaline or acid pH than at neutral pH.

9. The method of claim 1 where the amount of riboflavin ranges from 0.01 to 2% by weight of the dextran methacrylate.

10. The method of claim 1 where the electron donor is chitosan and the electron donating effective amount of chitosan ranges from 0.01 to 2 by weight of the dextran methacrylate.

11. A UV radiation initiated crosslinking hydrogel forming system comprising,
a) dextran methacrylate having a weight average molecular weight ranging from 30,000 to 200,000 grams per mole and a degree of substitution ranging from 0.08 to 0.60;
b) a photoinitiation effective amount of riboflavin ranging from 0.2 to 2% by weight of dextran methacrylate, and
c) an electron donating effective amount of L-arginine ranging from 0.8 to 1.2% by weight of dextran methacrylate.

12. A visible light initiated crosslinking hydrogel forming system comprising,
a) dextran methacrylate having a weight average molecular weight ranging from 30,000 to 200,000 grams per mole and a degree of substitution ranging from 0.08 to 0.60;
b) a photoinitiation effective amount of riboflavin ranging from 0.01 to 0.51% by weight of dextran methacrylate, and
c) an electron donating effective amount of L-arginine ranging from 1 to 20% by weight of dextran methacrylate.

13. A biodegradable nontoxic hydrogel including dextran as a polysaccharide moiety, which is free of residual synthetic photoinitiators.

14. The biodegradable nontoxic hydrogel of claim 13 which contains as residual photoinitiator only that which is activated by visible light.

15. The method according to claim 3 where the polysaccharide is dextran having a weight average molecular weight ranging from 30,000 to 200,000 grams per mole.
